(19) United States
(12) Patent Application Publication
(10) Pub. No.: US 2024/0102994 A1
(43) Pub. Date: Mar. 28, 2024

(54) Method to Evaluate the Capability of Compounds on the Trapping of Proteins

(71) Applicant: Eisbach Bio GmbH, Martinsried (DE)

(72) Inventors: Katharina Sahiri, Zorneding (DE); Charlotte Blessing, München (DE); Adrian Schomburg, Gräfelfing (DE); Andreas Ladurner, Gräfelfing (DE)

(21) Appl. No.: 18/039,742

(22) PCT Filed: Dec. 3, 2021
(86) PCT No.: PCT/EP2021/084088

§ 371 Date: Jun. 1, 2023

(30) Foreign Application Priority Data
Dec. 3, 2020 (EP) 20211716.4

(51) Int. Cl. G01N 33/50; G01N 33/58

(52) U.S. Cl. CPC G01N 33/5035; G01N 33/582; G01N 2333/91142; G01N 2333/914

(57) ABSTRACT

Screening methods as well as kits for identifying compounds capable of trapping proteins, e.g. proteins involved in DNA repair, are provided. The methods provide the use of live-cell imaging and local laser micro-irradiation of nuclear DNA in an assay to measure the effects of compounds on the trapping of 5 proteins on DNA. The disruption, e.g. inhibition, of specific proteins, such as poly-(ADP-ribose) polymerases or ALC1 enzyme, leads to trapping on chromatin and/or at DNA damage sites. This inhibits essential cellular functions, e.g. DNA damage repair, and can potentiate cancer cell killing.

### FIELD OF THE INVENTION

Methods are provided to identify compounds with capability of trapping proteins on DNA. The invention has application in the field of medicine and drug development. More particularly, this application relates to the use of live-cell imaging and local laser micro-irradiation of nuclear DNA in an assay to measure the effects of compounds on the trapping of proteins on DNA. The disruption of specific proteins, such as poly-(ADP-ribose) polymerases or ALC1 enzyme, leads to their trapping on chromatin and/or at DNA damage sites. This inhibits essential cellular functions, i.e. DNA damage repair, and can potentiate cancer cell killing. The method allows to directly measure the capability of compounds to trap proteins, e.g. at DNA lesions, and thus allows to determine their use as a protein-trapping compound. The method can further be used to examine the potentiation effect of drug candidates by measuring the extent of trapping upon co-treatment of the compound with further compound(s) and allows to monitor whether drugs can overcome resistance mechanisms that involve the trapping of proteins on DNA.

### BACKGROUND OF THE INVENTION

Cancer is the second most frequent cause of death in Europe, with 1.9 million deaths and over 3.7 million new cases each year (Luengo-Fernandez 2013). In Germany alone about 0.5 million people are each year diagnosed with cancer (Krebs in Deutschland 2015/2016). While sharing the phenotype of uncontrollable cell growth, cancer cells differ in their underlying mutations, making it a difficult-to-control disease. Conventional chemotherapeutics often target all rapidly dividing cells. This lack of specificity induces severe side effects, as healthy dividing cells are dying in addition to the targeted cancer cells.

The field of cancer medicine is therefore trying to shift to a more individualized cancer treatment. One emergent mechanism is the identification of mutated genes in cancer cells (typically deletions of established tumor suppressor genes, e.g. Breast cancer susceptibility protein (BRCA), Phosphatase and tensin homolog (PTEN) and subsequently treating the patient with a compound, e.g. a small-molecule drug that specifically targets the gene(s) that the tumor has become dependent on. A key goal of many researchers is thus the identification of specific genetic vulnerabilities (“Achilles heels”) of the cancer (see DepMap, Project Achilles, TCGA and Project Drive initiatives). Not only does this provide a cancer-cell-specific treatment with decreased side effects but also allows a more effective and dynamic treatment of patients. However, the number of targeted cancer treatments is still low, since many genetic dependencies remain untested and the development of new and more potent cancer drugs is urgently required.

The cancer genome is especially rich in mutations of DNA repair proteins, making the targeting of the cellular DNA damage response attractive for cancer therapy. DNA damage response ensures the repair of DNA lesions, meanwhile stalling replication and initiating cell death, if the damage is beyond repair.

Targeting DNA damage response (DDR) proteins for cancer therapy is shown by the approval of PARP inhibitors (PARPi) for use in the clinic. These small-molecule drugs target the poly-(ADP-ribose)-ribose proteins PARP1 and PARP2, which are required for efficient repair of single-strand and double-strand breaks in cells. Proteins of the PARP family are recruited to damage sites by recognizing specifically altered, DNA-damage induced structures. Their PARylation activity is triggered, which in turn regulates their own activity and the activity of other DDR-and chromatin proteins, facilitating mechanisms of damage repair (Ray Chaudhuri and Nussenzweig, 2017).

Poly(ADP-ribose)polymerases (PARP) enzymes are proteins involved in many processes in the cell. These cellular processes mainly involve DNA repair and apoptosis, programmed cell death. The PARP enzymes have the capacity to make a polymer of ADP-ribose (PAR) from nicotinamide adenine dinucleotide (NADH in its reduced form). Poly(ADP-ribose) polymerase-1 (PARP-1) is an example of a PARP enzyme which is able to bind damaged DNA and initiate the repair process upon recognition of DNA breaks caused by various genotoxic insults. Once bound to DNA, PARP-1 is activated and uses G-NAD+to poly(ADP-ribosyl)ate proteins such as histones, transcription factors, and itself, thus markedly altering the overall size and charge of the modified protein. Sites for poly(ADP-ribose) (PAR) binding have been identified in numerous DNA-damage checkpoint proteins including tumor suppressor p53, DNA-ligase III, X-ray repair cross-complementing 1 (XRCC1), DNA-dependent protein kinase (DNA-PK), NF-κB, and telomerase, consistent with the role of PAR in the DNA repair pathway. The cytoprotective role of PARP-1 in response to DNA damaging agents has been studied and is supported by experiments with PARP-1-deficient cell lines. Accordingly, inhibition of PARP-1 with small molecules has proven to potentiate anticancer drugs, and initial studies have demonstrated that some BRCA-1-deficient tumor cells are extremely sensitive to PARP-1 inhibition. Including PARP-1 there are several members in the PARP family of enzymes with significant biomedical relevance, and the ability to detect and measure the activity of such enzymes is of great interest. This catalytic activity can be inhibited by PARP inhibitors and has become of particular interest and clinically useful in genetically-defined cancers. The new trend of targeting PARP enzymes has emerged as a powerful tool for treatment of an increasing number of cancer-types. The approved PARPi exhibit large differences in clinical efficacy in killing cancer cells and have varying effects on patient outcomes in the clinic. One difference in PARPi-action lies in the promotion of distinct levels of PARP trapping on DNA/chromatin. The process of “trapping PARP” can be described as an enhanced retention of PARP enzymes on chromatin in living cells.

Current knowledge discloses PARPi that trap PARP1 at DNA damage sites as the most effective PARP inhibitors. The cytotoxic effect of PARP trapping especially affects cells that are deficient in repair of DNA strand breaks, such as homologous recombination (HR)-deficient BRCA1/2 mutant cancer cells, where DNA strand breaks exhibit a lethal effect. The mechanism of trapping in such cells is thought to lead to replicative stress as well as genomic instability leading to cell death (Lord and Ashworth, 2012). FIG. 1 shows the cytotoxic mechanism of PARP trapping via PARPi (modified from Murai et al., 2012).

Nevertheless, the power of PARP inhibitors, similarly to conventional cancer therapies, is limited by resistance mechanisms developing in tumor cells upon treatment. Long-term clinical studies indeed report that, albeit demonstrating treatment successes with low side effects, the majority of patients will develop resistance after prolonged treatment (Lheureux 2017). Resistant cancer cells have acquired additional mutations to re-activate double-strand break repair or to decrease cell cycle progression (Kondrashova 2017; Quigley 2017). These patients thus require new therapy options to battle the adapted cancer. The field of cancer medicine is therefore in need to both identify new biomarkers and develop additional inhibitors that allow alternative or combined treatment regiments in advanced, resistant tumors.

Pre-clinical research has shown that the recruitment and retention kinetics of several DNA repair proteins is critical to an efficient DNA damage response, and that especially the prolonged retention (=“protein trapping”) of proteins on chromatin interferes with cellular function (Puumalainen et al., 2014; Kalousi et al., 2015; Mok et al., 2019; Vohhodina et al., 2020). The search for potent and selective compounds is hampered by an inadequacy of suitable reagents and methods facilitating the detection and measurement of PARP enzymatic activity; moreover, there is a lack of high-throughput assays. Given the clinical success of PARPi, we propose that the chemical inhibition of such proteins may allow to develop new treatment regiments for various cancer types, and to overcome acquired resistance mechanisms. This may be achieved by direct trapping of a protein on chromatin or by enhancing or modulating the trapping of enzymes, such as PARP proteins, upon co-treatment with two drugs. Furthermore, this will allow the improved development of inhibitors to enable first line therapies in the DDR field as an alternative to PARPi. A clinical need also exists to improve the development of inhibitors to reduce off-target effects within the non-DNA damage-related PARP family of enzymes and with regard to other NAD-utilizing cellular factors, as well as to improve the development of inhibitors to reduce side effects of PARP inhibitors, such as neutropenia and other side-effects that contribute to tolerability and compliance of drug taking issues in cancer patients.

### SUMMARY OF THE INVENTION

The present invention provides a screening method to identify candidate compounds with capability to trap nuclear proteins on DNA. This assay can be used to identify inhibitors that induce protein trapping, and thus mediate sensitization of cells to this compound, bypass cancer therapeutic resistance mechanisms and/or promote cancer cell killing through a direct and/or indirect impact on protein trapping. Inhibitors can be distinguished that enhance PARP1, PARP2 and/or PARP3 trapping, as well as the trapping of other chromatin/DNA-damage associating proteins at DNA damage sites and thus promote replication stress, DNA damage and/or cancer cell killing. Inhibitors can be distinguished that enhance not only PARP trapping directly and/or indirectly through inhibition of another protein, it can also profile trapping of other DNA/RNA binding proteins, such as ATP-dependent chromatin remodeling enzymes or DNA damage-associated proteins at nuclear DNA damage sites, wherein the mechanism of trapping via those inhibitors promotes cellular cytotoxicity and cancer cell killing.

In one aspect, the invention provides a method for determining the capability of at least one compound for trapping of at least one protein of interest comprising the steps of
a. contacting a first cell comprising at least one detectably labelled protein with the at least one compound;
b. exposing said cell to conditions inducing damage to the cell's genetic material, suitably DNA and/or RNA;
c. detecting the at least one detectably labelled protein; and
d. identifying the at least one compound as capable of protein trapping when accumulation and/or residence time of the at least one protein of interest is increased;
   wherein the compound is selected from an inhibitor of at least one of chromodomain-helicase-DNA-binding protein 1-like (ALC1); DNA repair and DNA damage response protein from Table 1; DNA replication proteins from Table 2; chromatin remodelers defined by presence of a SNF2-family ATPase domain; histone chaperone; transcription factors; RNA binding proteins; proteins in PARP pathways from Table 3.
   In a second aspect the invention provides a kit comprising:
a first cell comprising at least one detectably labelled protein and a second cell comprising at least one detectably labelled protein; and
instructions for using the first cell and the second cell in a method for screening of a test compound for capability to trap a protein, wherein the test compound is selected from an inhibitor of at least one of inhibitor of at least one of chromodomain-helicase-DNA-binding protein 1-like (ALC1); DNA repair and DNA damage response protein from Table 1; DNA replication proteins from Table 2; chromatin remodelers defined by presence of a SNF2-family ATPase domain; histone chaperone; transcription factors; RNA binding proteins; proteins in PARP pathways from Table 3.

In a third aspect, the invention provides a screening assay comprising:
a. Exposing a plurality of cell samples to DNA damage conditions, wherein said samples comprise at least one protein; and
b. Determining protein trapping of the protein in a first cell sample in the presence of at least one test compound relative to a second cell sample not comprising said at least one test compound,
c. wherein a test compound contained in the first cell sample in which the detectably labelled protein exceeds the accumulation and/or residence time of the protein in the second cell sample is identified as a candidate compound for protein trapping,
   wherein said test compound is selected from an inhibitor of at least one of chromodomain-helicase-DNA-binding protein 1-like (ALC1); DNA repair and DNA damage response protein from Table 1; DNA replication proteins from Table 2; chromatin remodelers defined by presence of a SNF2-family ATPase domain; histone chaperone; transcription factors; RNA binding proteins; proteins in PARP pathways from Table 3.

### BRIEF DESCRIPTION OF THE FIGURES

FIG. 1: Cytotoxic mechanisms of PARPi on DNA repair pathways leading to PARP trapping

Lower pathway shows interference with DNA repair of single strand breaks (SSBs) via DNA replication fork damage leading to repair via the homologous recombination (HR) mechanism. Upper pathway shows trapping of PARP proteins (indicated by black hexagon) on damaged DNA, leading to replication fork damage stalling. Additional repair pathways are needed, including Fanconi pathway (FA), template switching (TS), ATM, FEN1 (replicative flap endonuclease) and DNA polymerase 13 (modified from Murai et al., 2012).

FIG. 2: Schematic of live-cell protein trapping assay

The cell nucleus is micro-irradiated by a UV laser to induce DNA damage (indicated by black bar, left cell). Cells are then imaged for a determined time frame (e.g. 5-30 minutes). The black bar indicates the recruitment of the fluorophore-tagged protein to the induced DNA damage site (middle cell). A) the signal of protein engaged with the damage reduces over time, indicating a decrease in protein retention B) prolonged retention of the protein upon drug treatment, showing molecular trapping of the inhibited protein at the induced DNA damage site.

FIG. 3: Example of protein trapping: GFP-PARP2 association at laser micro-irradiation sites in U2OS cells

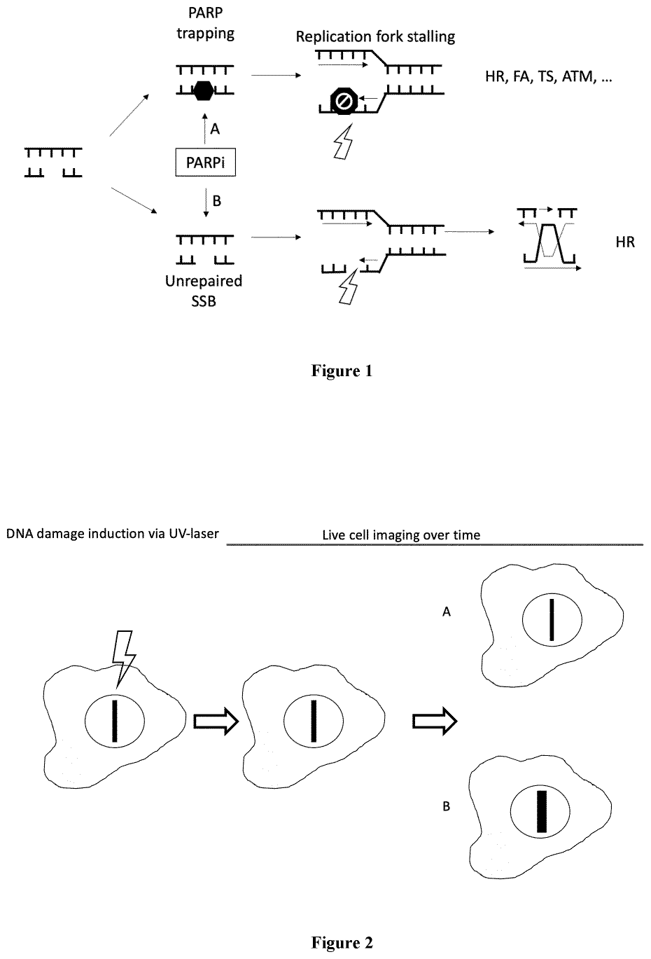

U2OS cells were treated with PARPi veliparib (10 μM) and talazoparib (100 nM) and transfected with GFP-PARP2. Grey signal indicates GFP-PARP2 in the nucleus. Kinetics of GFP-PARP2 recruitment to and dissociation from DNA lesion (induced by a 10% 355 nm laser) was measured over 15 minutes in the presence and absence of PARPi. Bright lines show recruitment of PARP2 to laser micro-irradiated damage sites. Images were taken 1 minute and 15 minutes after irradiation. Treatment with talazoparib shows enhanced retention of PARP2 at induced damage sites (“PARP trapping”), whereas treatment with veliparib leads to less recruitment of PARP2 to the damage site.

FIG. 4: Relative recruitment of GFP-ALC1 to DNA damage site after treatment with an ALCi inhibitor

Wild-type U2OS cells were transiently transfected with GFP-ALC1. Kinetics of GFP-ALC1 recruitment to and dissociation from DNA lesion (induced by a 10% 355 nm laser) was measured over 5 minutes in the presence and absence of compound ALCi-1 (50 μM). Treatment with the compound shows enhanced retention of ALC1 at DNA damage sites compared to DMSO. 10 nuclei were analyzed in 1 biological replicate. The data are shown as mean+S.E.M. normalized to pre-damaged GFP intensity at micro-irradiation sites.

FIG. 5: Relative recruitment of GFP-PARP2 to DNA damage site after treatment with an ALCi inhibitor

Wild-type U2OS cells were transiently transfected with GFP-PARP2. Kinetics of GFP-PARP2 recruitment to and dissociation from DNA lesion (induced by a 15% 355 nm laser) was measured over 30 minutes in the presence and absence of ALCi-2 (10 μM). Treatment with ALCi-2 shows enhanced retention of PARP2 at DNA damage sites compared to DMSO. 5-15 nuclei were analyzed in 1 biological replicate. The data are shown as mean+S.E.M. normalized to pre-damaged GFP intensity at micro-irradiation sites.

FIG. 6: Images of relative recruitment of GFP-PARP2 to DNA damage site after treatment with an ALC inhibitor

Wild-type U2OS cells were transiently transfected with GFP-PARP2. Kinetics of GFP-PARP2 recruitment to and dissociation from DNA lesion (induced by a 15% 355 nm laser) was measured over 30 minutes in the presence and absence of ALCi-2 (10 μM). Upper nucleus was treated with DMSO, lower nucleus was treated with ALCi-2 (10 μM) for 1 h. Timepoint 0 min. shows nuclei before micro-irradiation, timepoint 1 min. and 30 min. show nuclei after irradiation. Treatment with ALCi shows PARP2-trapping at DNA damage sites compared to DMSO.

FIG. 7: PARP2 trapping after co-treatment with ALCi and PARPi veliparib

Wild-type U2OS cells were transiently transfected with GFP-PARP2. Kinetics of GFP-PARP2 recruitment to and dissociation from DNA lesion (induced by a 10% 355 nm laser) was measured over 15 minutes in the presence and absence of veliparib (10 μM) or a combination of veliparib (10 μM) and ALCi-2 (10 μM). 4-11 nuclei were analyzed in 1 biological replicate. The data are shown as mean+S.E.M. normalized to pre-damaged GFP intensity at micro-irradiation sites.

FIG. 8: PARP1 trapping after treatment with different PARPi

Wild-type U2OS cells were transiently transfected with PARP1-GFP. Kinetics of PARP1-GFP recruitment to and dissociation from DNA lesion (induced by a 15% 355 nm laser) was measured over 30 minutes in the presence and absence of olaparib (10 μM), veliparib (10 μM) or talazoparib (100 nM). Treatment with PARPi for 1 h shows enhanced retention of PARP1 at DNA damage sites compared to DMSO. 5-20 nuclei were analyzed in 2 biological replicates. The data are shown as mean+S.E.M. normalized to pre-damaged GFP intensity at micro-irradiation sites.

FIG. 9: Images of relative recruitment of PARP1-GFP to DNA damage site after treatment with different PARP inhibitors

Wild-type U2OS cells were transiently transfected with PARP1-GFP. Kinetics of PARP1-GFP recruitment to and dissociation from DNA lesion (induced by a 15% 355 nm laser) was measured over 30 minutes in the presence and absence of PARPi for 1 h (Olaparib (10 μM), Veliarpib (10 μM), Talazoparib 100 nM). Upper nucleus was treated with DMSO. Timepoint 0 min. shows nuclei before micro-irradiation, timepoint 1 min. and 30 min. show nuclei after irradiation. Treatment with PARPi shows PARP1-trapping at DNA damage sites compared to DMSO.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

The inventors have identified general techniques for identifying the capability of a compound to trap proteins. The identification of compounds capable of trapping proteins can aid in the identification of suitable compounds for use in treatment of certain diseases, e.g. cancer.

Provided herein is a method for determining the capability of at least one test compound for trapping of at least one protein of interest comprising the steps of
a. contacting a first cell comprising at least one protein, optionally wherein said at least one protein is detectably labelled, with the at least one compound;
b. exposing said cell to conditions inducing damage to the cell's genetic material, suitably DNA and/or RNA;
c. detecting at least one protein, optionally wherein said at least one protein is detectably labelled; and
d. identifying the at least one compound as capable of protein trapping when the accumulation and/or residence time of the at least one protein is increased;
   wherein the test compound is selected from an inhibitor of at least one of chromodomain-helicase-DNA-binding protein 1-like (ALC1); DNA repair and DNA damage response protein from Table 1; DNA replication proteins from Table 2; chromatin remodelers defined by presence of a SNF2-family ATPase domain; histone chaperone; transcription factors; RNA binding proteins; proteins in PARP pathways from Table 3. Suitably, the protein is chromodomain-helicase-DNA-binding protein 1-like (ALC1). Said method may further comprise contacting a second cell comprising at least one protein, optionally wherein said at least one protein is detectably labelled, with a control compound, wherein the at least one test compound is considered capable of protein trapping when the accumulation and/or residence time of the at least one protein is higher in the first cell than in the second cell. Suitably, the at least one detectable protein and the at least one inhibited protein are the same. Suitably, the at least one detectable protein and the at least one inhibited protein are different.

Also provided herein is a kit comprising a first cell comprising at least one protein, optionally wherein said at least one protein is detectably labelled, and a second cell comprising at least one protein, optionally wherein said at least one protein is detectably labelled; and instructions for using the first cell and the second cell in a method for screening of a test compound for capability to trap a protein of interest, wherein the test compound is selected from an inhibitor of at least one of chromodomain-helicase-DNA-binding protein 1-like (ALC1); DNA repair and DNA damage response protein from Table 1; DNA replication proteins from Table 2; chromatin remodelers defined by presence of a SNF2-family ATPase domain; histone chaperone; transcription factors; RNA binding proteins; proteins in PARP pathways from Table 3. Suitably, the protein is chromodomain-helicase-DNA-binding protein 1-like (ALC1). Said method of screening comprises contacting a first cell comprising at least one protein, optionally wherein said at least one protein is detectably labelled, with a test compound and contacting a second cell comprising at least one protein, optionally wherein said at least one protein is detectably labelled, with a control compound, wherein the at least one compound is considered capable of protein trapping when the accumulation and/or residence time of the at least one protein is higher in the first cell than in the second cell.

Also provided herein is a screening assay comprising exposing a plurality of cell samples to DNA damage conditions, wherein said samples comprise at least one protein, optionally wherein said at least one protein is detectably labelled, and determining protein trapping of the at least one protein in a first cell sample in the presence of at least one test compound relative to a second cell sample not comprising said at least one test compound, wherein a test compound contained in the first cell sample in which the protein exceeds the accumulation and/or residence time of the protein in the second cell sample is identified as a candidate compound for protein trapping, wherein said test compound is an inhibitor of Chromodomain-helicase-DNA-binding protein 1-like (ALC1).

Suitably, the protein of interest is selected chromodomain-helicase-DNA-binding protein 1-like (ALC1); DNA repair and DNA damage response protein from Table 1; DNA replication proteins from Table 2; chromatin remodelers defined by presence of a SNF2-family ATPase domain; histone chaperones; transcription factors; RNA binding proteins; proteins in PARP pathways from Table 3. Suitably, the protein is chromodomain-helicase-DNA-binding protein 1-like (ALC1).

TABLE 1
DNA repair and DNA damage response protein
Gene Name (synonyms)

Base excision repair (BER)
UNG
SMUG1
MBD4
TDG
OGG1
MUTYH (MYH)
NTHL1 (NTH1)
MPG
NEIL1
NEIL2
NEIL3
Other BER and strand break joining factors
APEX1 (APE1)
APEX2
LIG3
XRCC1
PNKP
APLF
HMCES
Poly(ADP-ribose) polymerase (PARP) enzymes that bind to DNA
PARP1 (ADPRT)
PARP2 (ADPRTL2)
PARP3 (ADPRTL3)
PARG
PARPBP
Direct reversal of damage
MGMT
ALKBH2 (ABH2)
ALKBH3 (DEPC1)
Repair of DNA-protein crosslinks
TDP1
TDP2 (TTRAP)
SPRTN (Spartan)
Mismatch excision repair (MMR)
MSH2
MSH3
MSH6
MLH1
PMS2
MSH4
MSH5
MLH3
PMS1
PMS2P3 (PMS2L3)
HFM1
Nucleotide excision repair (NER)
XPC
RAD23B
CETN2
RAD23A
XPA
DDB1
DDB2 (XPE)
RPA1
RPA2
RPA3
ERCC3 (XPB)
ERCC2 (XPD)
GTF2H1
GTF2H2
GTF2H3
GTF2H4
GTF2H5 (TTDA)
GTF2E2
CDK7
CCNH
MNAT1
ERCC5 (XPG)
ERCC1
ERCC4 (XPF)
LIG1
ERCC8 (CSA)
ERCC6 (CSB)
UVSSA (KIAA1530)
XAB2 (HCNP)
MMS19
Homologous recombination
RAD51
RAD51B
RAD51D
HELQ (HEL308)
SWI5
SWSAP1
ZSWIM7 (SWS1)
SPIDR
PDS5B
DMC1
XRCC2
XRCC3
RAD52
RAD54L
RAD54B
BRCA1
BARD1
ABRAXAS1
PAXIP1 (PTIP)
SMC5
SMC6
SHLD1
SHLD2 (FAM35A)
SHLD3
SEM1 (SHFM1) (DSS1)
RAD50
MRE11A
NBN (NBS1)
RBBP8 (CtIP)
MUS81
EME1 (MMS4L)
EME2
SLX1A (GIYD1)
SLX1B (GIYD2)
GEN1
Fanconi anemia
FANCA
FANCB
FANCC
BRCA2 (FANCD1)
FANCD2
FANCE
FANCF
FANCG (XRCC9)
FANCI (KIAA1794)
BRIP1 (FANCJ)
FANCL
FANCM
PALB2 (FANCN)
RAD51C (FANCO)
SLX4(FANCP)
FAAP20 (C1orf86)
FAAP24 (C19orf40)
FAAP100
UBE2T (FANCT)
Non-homologous end-joining
XRCC6 (Ku70)
XRCC5 (Ku80)
PRKDC
LIG4
XRCC4
DCLRE1C (Artemis)
NHEJ1 (XLF, Cernunnos)
Modulation of nucleotide pools
NUDT1 (MTH1)
DUT
RRM2B (p53R2)
PARK7 (DJ-1)
DNPH1
NUDT15 (MTH2)
NUDT18 (MTH3)
DNA polymerases
POLA1
POLB
POLD1
POLD2
POLD3
POLD4
POLE (POLE1)
POLE2
POLE3
POLE4
REV3L (POLZ)
MAD2L2 (REV7)
REV1 (REV1L)
POLG
POLH
POLI (RAD30B)
POLQ
POLK (DINB1)
POLL
POLM
POLN (POL4P)
PRIMPOL
DNTT
Editing and processing nucleases
FEN1 (DNase IV)
FAN1 (MTMR15)
TREX1
TREX2
EXO1 (HEX1)
APTX (aprataxin)
SPO11
ENDOV
DNA2
DCLRE1A (SNM1A)
DCLRE1B (SNM1B)
EXO5
Ubiquitination and modification
UBE2A (RAD6A)
UBE2B (RAD6B)
RAD18
SHPRH
HLTF (SMARCA3)
RNF168
RNF8
RNF4
UBE2V2 (MMS2)
UBE2N (UBC13)
USP1
WDR48
HERC2
Chromatin Structure and Modification
H2AX (H2AFX)
CHAF1A (CAF1)
SETMAR (METNASE)
ATRX
Genes defective in diseases associated with sensitivity to DNA damaging agents
BLM
RMI1
TOP3A
WRN
RECQL4
ATM
MPLKIP (TTDN1)
Other identified genes with known or suspected DNA repair function
RPA4
PRPF19 (PSO4)
RECQL (RECQ1)
RECQL5
RDM1 (RAD52B)
NABP2 (SSB1)
Other conserved DNA damage response genes
ATR
ATRIP
MDC1
PCNA
RAD1
RAD9A
HUS1
RAD17 (RAD24)
CHEK1
CHEK2
TP53
TP53BP1 (53BP1)
RIF1
TOPBP1
CLK2
PER1
translesion synthesis (TLS) polymerases, such as REV1, POL η, POL ι, POL κ, POL ζ, POL μ, POL λ, POL
β, POL ν, POL θ
topoisomerase enzymes, such as TOP I, TOP II, TOP III
AP endonucleases, such as APE1, APE2
polynucleotide kinase/phosphatase (PNKP)
tyrosyl DNA phosphodiesterase 1 (TDP1)
O6- alkylguanine-DNA alkyltransferase (AGT/MGMT) enzyme
AlkB-related α-ketoglutarate-dependent dioxygenases, such as (AlkB) A (AlkbH1-8), FTO
DNA glycosylases, such as N-methylpurine DNA Glycosylase (MPG), and MutY Homolog (MUTYH),
DNA glycosylase 1 (NTHL1), Nei-like DNA glycosylase 1 (NEIL1) and Nei-like DNA glycosylase 2
(NEIL2), 8-oxoguanine DNA glycosylase (OGG1), NEIL3
POL β, POL δ, POLε
LIG1 (DNA ligase 1)
LIG3 (DNA ligase 3)
XRCC1 (X-ray repair cross-complementing protein 1)
PARP2
PARP3
XPC (Xeroderma Pigmentosum, complementation group C), RAD23B (UV excision repair protein
Radiation sensitive 23B), CETN2 (Centrin 2)
DDB1 (XPE-binding factor), DDB2
transcription initiation factor II H (TFIIH) complex
XPF-ERCC1, XPG
PCNA (proliferating cell nuclear antigen), RFC (replication factor C)
RNA Polymerase II
CSA (Cockayne syndrome WD repeat protein A), CSB (Cockayne syndrome protein B)
UVSSA (UV-stimulated scaffold protein A), USP7 (ubiquitin-specific-processing protease 7), XAB2
(XPA-binding protein 2), HMGN1 (high mobility group nucleosome-binding domain-containing protein 1
MSH2, MSH6, MSH3; MutL, MutS, MLH1, PMS2, EXO1 (exonuclease 1)
Fanconi Anemia proteins, such as FANCA, FANCB, FANCC, FANCD1, FANCD2, FANCE, FANCF,
FANCG, FANCI, FANCJ, FANCL, FANCM, FANCN, FANCO, FANCP, FANCQ, FANCR, FANCS,
FANCT, FANCU, FANCV, FAAP24 (Fanconi Anemia associated protein of 24 kDa), FAAP24 (Fanconi
Anemia associated protein of 24 kDa)
RPA
MUS8-EME1, SLX4-SLX1, FAN1, SNM1A/SNM1B
CtIP
aprataxin (APTX)
FEN1
MDC1, 53BP1, BRCA1, BRCA2, Ku70, Ku80, DNA-PKcs, XRCC4, LIG4, XLF (XRCC4-like factor),
APLF (Aprataxin-and-PNK-like factor), TdT (terminal deoxynucleotidyl transferase)
MRE11, RAD50, NBS1, ATM, ATR, CHK1, CHK2, ATRIP, TopBP1, TIP60, RNF8, RNF168
HELLS
RAD51, RAD54, RAD54B
BLM, DNA2, PALB2
SMAD4
BLM-TOPOIII-RMI1-RMI2 complex, GEN1 endonuclease, the MUS81-EME1 complex and the SLX1-
SLX4 complex
sheltrin protein POT1 (protection of telomeres 1)
TRF1 (telomeric-repeat binding factor 1), TRF2, TIN2 (TRF-interacting protein 2), the transcriptional
repressor/activator protein RAP1, and the TPP1 (POT1- and TIN2- organizing protein)
DNMT1, DNMT3A, DNMT3B
METTL3, METTL14,

TABLE 2
DNA replication proteins

AND1
Cdc45
Cdc45-Mcm-GINS (CMG) complex
Cdc6
Cdc7-Dbf4 kinase
Cdt1
Claspin
Ctf4
Cyclin-dependent kinase (CDK)
Dna2
DNA ligase I
DNA polymerase α (Pol α)
DNA polymerase δ (Pol δ)
DNA polymerase ε (Pol ε)
Dpb11
Fen1
Geminin
GINS, Sld5, Psf1, Psf2, Psf3
Minichromosome maintenance proteins (Mcm), such as Mcm2-7
Mcm10
Mrc1
Origin recognition complex (ORC), such as Orc1-Orc6 proteins
Proliferating cell nuclear antigen (PCNA)
Replication factor C (RFC)
Replication fork barriers (RFBs)
Replication protein A (RPA)
RNase H
Sld2
Sld3
Telomerase
Topoisomerases

TABLE 3
Proteins of PARP pathway

PARP2
PARP3
poly(ADP-ribose) glycohydrolase (PARG)
ADP-ribosylhydrolase 3 (ARH3)
MacroD1, MacroD2 and TARG1
PARP7, PARP9, PARP10; PARP11, PARP12, PARP13, PARP14,
PARP15
macroH2A

Suitably, the protein is selected from Table 1; Table 2; Chromatin remodelers defined by SNF2-family ATPase and their interacting partners, e.g. see https://www.ncbi.nlm.nih.gov/pmc/articles/PMC503 9004/, which is hereby incorporated in its entirety; histone chaperones; transcription factors, e.g. see https://en.wikipedia.org/wiki/List_of humantranscription_factors, which is hereby incorporated in its entirety; RNA binding proteins, e.g. see hilp://rbpdb.ccbr.utoronto.ca/proteins.php?species_filter=9606; Proteins in PARP pathway from Table 3 and chromodomain-helicase-DNA-binding protein 1-like (ALC1).

The detectable label suitably can be a fluorescent label and/or a quantum dot label. The damage to the cell's genetic material, suitably DNA and/or RNA, suitably DNA, can be induced by any method known in the art. Suitably, the DNA damage can be induced by laser irradiation, for example by UV micro-irradiation or by multiphoton laser irradiation. Other examples of the DNA damaging agent include radiation, UV radiation, oxygen radicals, and hydrocarbons, but are not limited thereto. Other examples of DNA damage can be indice by agents that are known to cleave the DNA at certain sites to induce single- or double strand breaks. Such agents are, for example, transcription activator-like effector nucleases (TALEN), zinc-finger nucleases (ZFN), CRISPR/Cas9, CRISPR/nickase, megaendonucleases, NgAgo and chimeric nucleases. The detectably labelled protein can be detected by live cell imaging or by fixed cell imaging, suitably live cell imaging. The protein can be selected from DNA-binding proteins; DNA damage factors involved in direct damage reversal, base excision repair, nucleotide excision repair, DNA mismatch repair, inter-strand crosslink repair, homologous recombination, non-homologous end joining; proteins involved in DNA replication; RNA-binding proteins; PARP proteins (PARP1, PARP2, PARP3); Proteins involved in the poly-ADP-ribose response; chromatin remodeling proteins; Chromodomain-helicase-DNA-binding protein 1-like (ALC1); ataxia telangiectasia and Rad3-related protein (ATR); ataxia telangiectasia mutated (ATM); RAD51; Pol Theta. Suitably, the protein of interest is PARP1, suitably PARP2, suitably PARP3, suitably ALC1. Suitably, the detectably labelled protein of interest is PARP1. Suitably, the detectably labelled protein of interest is PARP2. Suitably, the detectably labelled protein of interest is PARP3. Suitably, the detectably labelled protein of interest is ALC1. Suitably, the first and/or second cell is a eukaryotic cell. Suitably, the cell is isolated. Suitably the cell is a single cell or a population of cells. Suitably, the first and/or second cell is a human cell. Suitably, the first cell is a human cell. Suitably, the second cell is a human cell. Suitably, the human cell can be a primary cell or it can be a cell derived from a human cell line. For example, human cell lines are commercially available, e.g. from a vendor, e.g. the American Type Culture Collection or the human cell line may not be from a commercial source. The primary cell can be obtained from a biopsy specimen. The cells may be transfected with a protein of interest, suitably which is detectably labelled, and expression of this protein may be constitutive or under regulatory control, e.g., inducible expression. The transfection vector may be a plasmid, virus, or any other suitable vector. Alternatively, or in addition, an endogenous protein may be modified to be detectably labelled, e.g. through insertion of a respective coding sequence for a label. Such methods are known in the art. Any cell that is amenable to manipulation to the methods disclosed herein can be used. For example, the cells can be U2OS cells. The first and second cells can have the same origin, e.g. they can be from the same sample, or they can be from a different sample. Suitably, the first and second cells are from the same sample. By way of example but not of limitation, where U2OS cells are used in the assay, both the first cell and the second cell are U2OS cells expressing a protein of interest, suitably one which is detectably labelled. Suitably, the protein trapping is direct and/or indirect. Suitably, the protein trapping is direct. Suitably, the protein trapping is indirect. Suitably, the protein trapping is direct and indirect. Suitably, the cells are contacted with at least two compounds. Suitably, the first compound is an ALC1 inhibitor and the second compound is a PARP inhibitor. Suitably, the first compound is an ALC1 inhibitor and the second compound is a PARP inhibitor selected from talazoparib, veliparib, olaparib, pamibarip, rucaparib, niraparib, CEP-9722, E7016. Suitably, the method can be used to determine the synergistic effect of at least two compounds on protein trapping. The synergistic effect can be on a protein directly binding the cell's genetic material, e.g. DNA and/or RNA, and/or it can be on a protein indirectly binding the cell's genetic material, e.g. DNA and/or RNA.

Various terms relating to aspects of disclosure are used throughout the specification and claims. Such terms are to be given their ordinary meaning in the art, unless otherwise indicated. Other specifically defined terms are to be construed in a manner consistent with the definition provided herein.

All patents, published patent applications, publications, references and other material referred to herein are incorporated by reference herein in their entirety.

It will be understood that unless indicated to the contrary, terms intended to be “open” (e.g., the term “including” should be interpreted as “including but not limited to,” the term “having” should be interpreted as “having at least,” the term “includes” should be interpreted as “includes but is not limited to,” etc.). Accordingly, the term “comprising” encompasses “including” as well as “consisting essentially of” and “consisting of” e.g. a composition “comprising” X may consist exclusively of X or may include something additional, e.g., X+Y.

Phrases such as “at least one,” and “one or more,” and terms such as “a” or “an” include both the singular and the plural.

The term “or” is used herein to mean, and is used interchangeably with, the term “and/or”, unless context clearly indicates otherwise.

It is understood that the methods of the invention disclosed herein are carried out ex vivo.

As used herein the term “protein trapping” and derivatives thereof refers to the capability of a compound, suitably a test compound, to increase the recruitment and/or retention and/or residence time and/or accumulation and/or enhanced binding, e.g. to DNA and/or RNA at sites of damage, of at least one protein, e.g. DNA-binding proteins; DNA damage factors involved in direct damage reversal, base excision repair, nucleotide excision repair, DNA mismatch repair, inter-strand crosslink repair, homologous recombination, non-homologous end joining; proteins involved in DNA replication; RNA-binding proteins; PARP proteins (PARP1, PARP2, PARP3); Proteins involved in the poly-ADP-ribose response; chromatin remodeling proteins; Chromodomain-helicase-DNA-binding protein 1-like (ALC1); ataxia telangiectasia and Rad3-related protein (ATR); ataxia telangiectasia mutated (ATM); RAD51; Pol Theta.

For example, by blocking access of NAD+ to the catalytic site, PARPi lock PARP1 in an inactive cytotoxic conformation, a situation generally referred to as “PARP trapping”. By trapping the protein, the accumulation and/or residence time on the cell's genetic material, suitably DNA and/or RNA, is increased.

As used herein, the term “residence time” refers to the amount of time a protein, e.g. one which is detectably labelled, is detected at or near a site of damage, e.g. DNA and/or RNA damage.

As used herein, the term “inhibiting” and derivatives thereof, e.g. “inhibitor”, comprises reducing, decreasing, blocking, preventing, delaying, inactivating, desensitizing, stopping, and/or downregulating activity or expression of a molecule or pathway of interest. By way of example, but not of limitation, inhibition of a PARP family member protein, such as PARP1, includes inhibiting its enzymatic activity, including the ability to bind damaged DNA and initiate the repair process upon recognition of DNA breaks. Inhibiting can, but need not be 100%.

“Label” and “detectable label” refers to a moiety attached to a protein of interest to render the protein detectable, and the protein so labeled is referred to as “detectably labeled.” A label can produce a signal that is detectable by visual or instrumental means. Various labels include signal-producing substances, such as chromogens, fluorescent compounds, chemiluminescent compounds, radioactive compounds, quantum dots and the like. Representative examples of labels include moieties that produce fluorescence, e.g., green-fluorescent protein (GFP). In this regard, the moiety, itself, may not be detectable but may become detectable upon reaction with yet another moiety. Use of the term “detectably labeled” is intended to encompass such labeling.

As used herein, the term “detect” refers to the act of viewing, imagining, indicating the presence of, measuring, and the like of at least one protein of interest based on the emission, e.g. light, fluorescence etc. from the label. More specifically, in some instances the presently labelled proteins can be bound and/or fused to a label, e.g. a fluorescent label, a quantum dot label or another label, and, upon being exposed to an absorption light, will emit an emission light. The presence of an emission light can indicate the presence of a protein, whereas the quantification of the light intensity can be used to measure the concentration of a protein. Determining or detecting may comprise any suitable quantitative or qualitative technique for measurement. For example, detecting may be performed by live cell imaging using a suitable instrument, such as a microscope. Detecting may be performed using any suitable method to detect the presence of a protein at or near the site of interest, e.g. at or near the site of DNA damage, for example, but not limited to, using FRAP (fluorescence recovery after photobleaching), Fluorescence Lifetime Imaging (FLIM), quantitative phase contrast microscopy, holotomography, Single-molecule localization microscopy (SMLM).

As used herein, the term “nucleic acid” refers to polynucleotides such as deoxyribonucleic acid (DNA), and, where appropriate, ribonucleic acid (RNA). The term should also be understood to include, as applicable to the embodiment being described, single-stranded (such as sense or antisense) and double-stranded polynucleotides.

Assays can be designed to measure the accumulation and/or residence time of at least one protein of interest, suitably one which is detectably labelled, at a site of damage on the cell's genetic material, e.g. DNA and/or RNA. The assays can be designed to determine the recruitment and/or retention of a protein. Assays can be designed to measure the accumulation and/or residence time at multiple time points to determine the level of label per unit time and/or under varying conditions. The screening assay can be carried out in any of a variety of formats. For example, for example the assay can be carried out in multiwell plates. In one approach, automated (robotic) handling of reagents and samples can be used. Multiwell plates microtiter plates are available in several format including 96-well plates, 384-well plates and 1,536-well plates.

In the screening assay a plurality of test samples can be screened simultaneously. A screening assay will often include multiple test samples with the same test agent (duplicates and/or at different concentration) as well as reference samples.

In addition to test samples, assays can include reference, or “control”, samples. For example, one reference sample can contain water or DMSO but no test compound and is otherwise processed in the same manner as test samples (e.g. including the damage step). The accumulation and/or residence time of at least one protein, suitably which is detectably labelled, in this reference provides the baseline against which accumulation and/or residence time of at least one protein, suitably which is detectably labelled, in test samples is compared. A test compound contained in a test sample in which accumulation and/or residence time of at least one protein of interest, suitably which is detectably labelled, exceeds the accumulation and/or residence time of at least one protein of interest, suitably which is detectably labelled, in the reference sample is identified as a candidate compound for protein trapping. Another type of reference sample contains a test compound, but the genetic material, e.g. the DNA and/or RNA, of the cell in the test sample is not damaged. Another type of reference sample contains no test compound and the genetic material, e.g. the DNA and/or RNA, of the cell in the test sample is not damaged. Test compounds that are capable of protein trapping can be distinguished from compounds that have no protein trapping capability by comparing the accumulation and/or residence time of the protein of interest, suitably which is detectably labelled, of the test sample and reference sample(s).

A suitable cell line in the methods disclosed herein is the osteosarcoma cell line termed U2OS. The U2OS cell line is a human cancer cell line that was established from a 15-year-old, Caucasian female in 1964 by J. Ponten and E. Saksela from a moderately differentiated sarcoma of the tibia. (U-2 OS ATCC 0 HTB-96™ Homo sapiens bone osteosarcoma, 2016) It is available from numerous sources including ATCC® HTB-96®.

Thus, next to U2OS cells, many other cell lines can be used for the method, including cell lines like SUM-149-PT, MDA-MB-231, HeLa, VeroE6, MCF7, MCF10a, PC-3, 22RV1, MEF and many more. Other cell-types like iPSC, primary cells, stem cells available on the ATCC platform can also be used with the method.

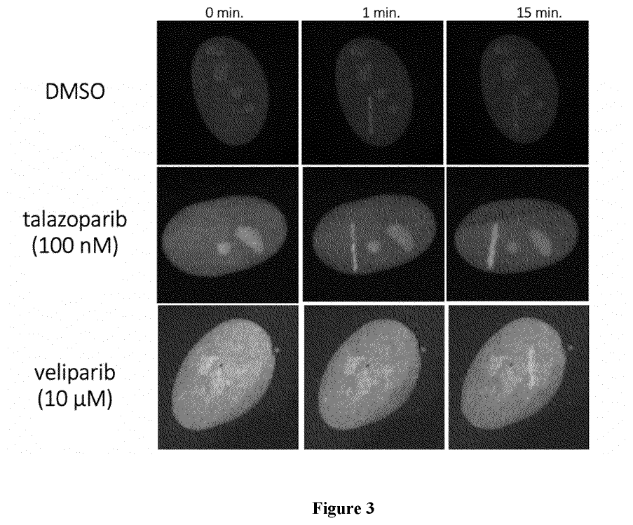

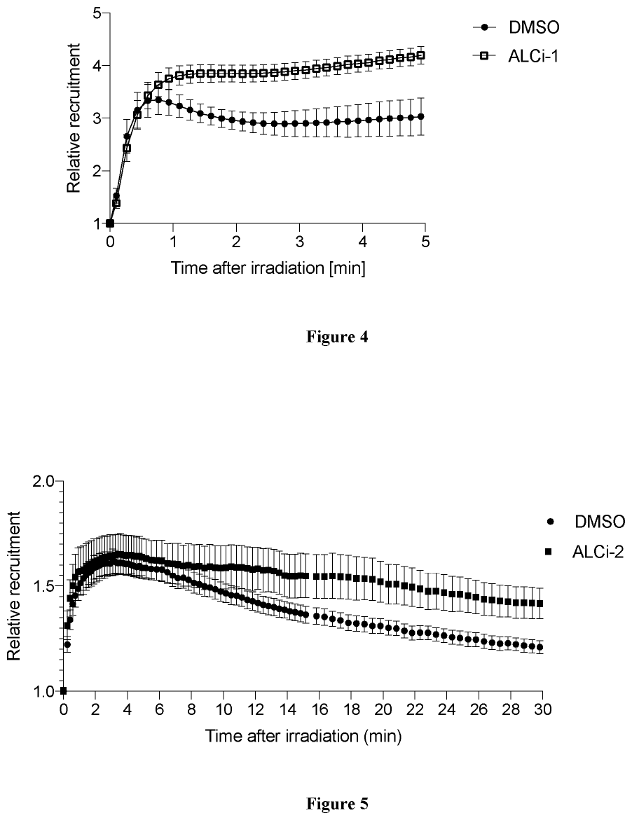

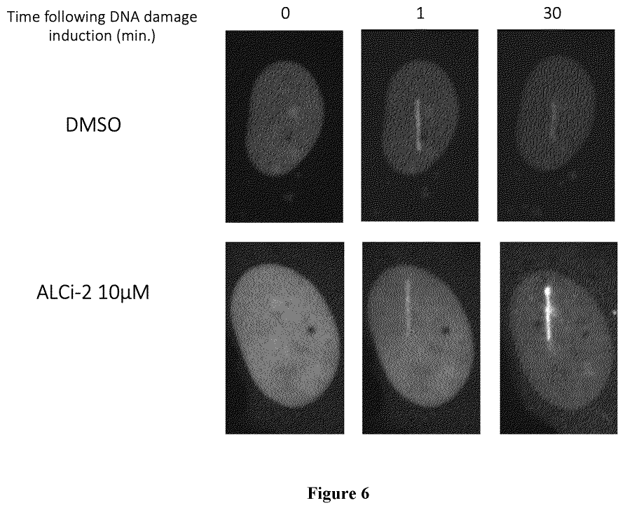

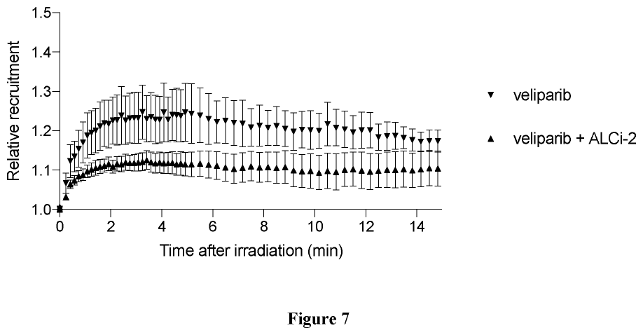

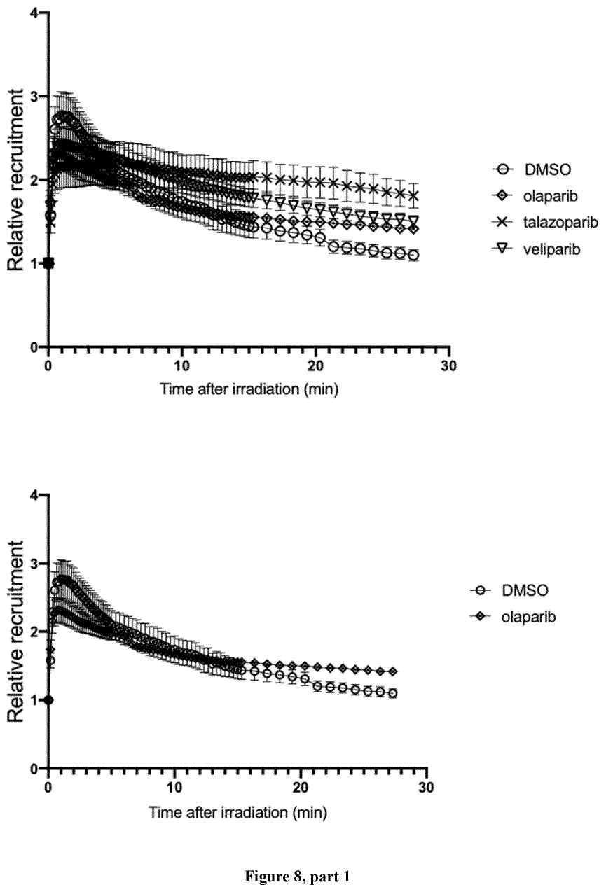

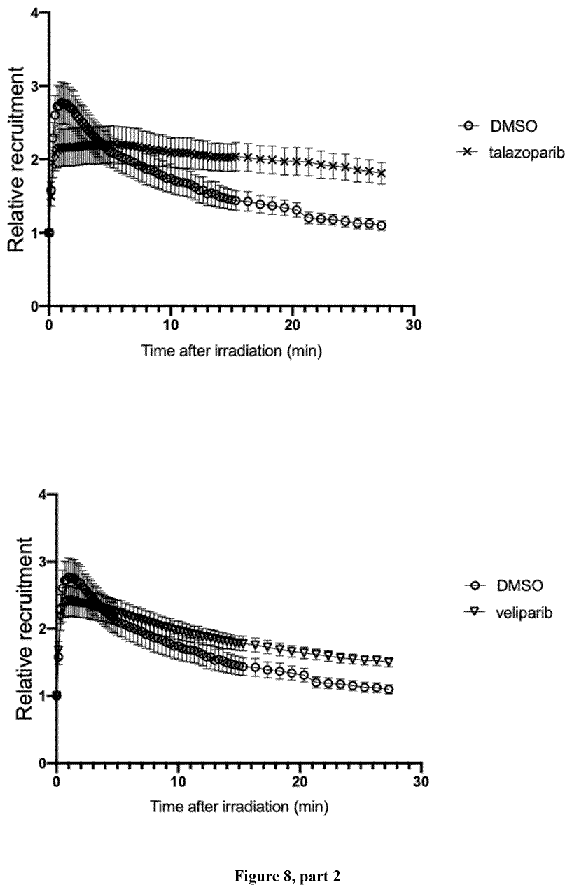

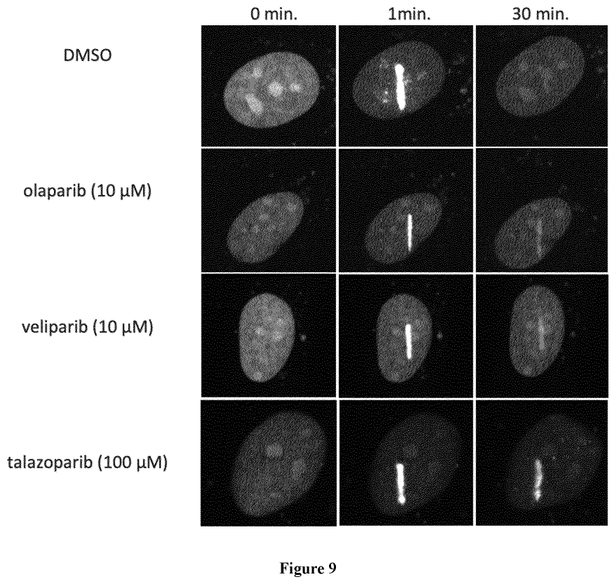

The dynamics of protein enrichment on chromatin can be visualized using live-cell imaging, when coupled to micro-irradiation with irradiation that site-specifically induces DNA lesions in the cell nucleus (Ahel et al., 2009; Gottschalk et al., 2009). The disclosed method is used to analyze protein trapping induced by compounds. Live-cell imaging allows monitoring of the accumulation of at least one protein, e.g. which is detectably labelled, e.g. fluorophore-tagged, at the induced DNA lesions over time. Time0 (t0) hereby describes the timepoint before induction of DNA damage by irradiation. Thus, the relative enrichment of the at least one protein at DNA lesions within the imaged time frame (from t0 to tEND) can be quantified. This allows to the analysis of the recruitment and retention (protein trapping) kinetics of the at least one protein, e.g. which is detectably labelled, e.g. fluorophore-tagged, at DNA lesions. Suitably, as a first step the accumulation and/or residence time of a protein, e.g. which is detectably labelled, e.g. fluorophore-tagged, is measured in the absence of a test compound (control). The optimal imaging time is hereby determined by the time in which the majority of the protein has released from the damage site, indicated by the decrease of signal, e.g. fluorescent signal, at the DNA lesion (see FIG. 2A). Subsequently, the accumulation and/or residence time of the protein, e.g. which is detectably labelled, e.g. fluorophore-tagged, may be determined in the presence of at least one test compound. If protein trapping is observed in the presence of the at least one test compound, the signal, e.g. fluorescent signal, does not decrease or suitably to a lesser extent than the control within the determined time frame (see FIG. 2B). Different mechanisms of PARPi on PARP2 trapping (see FIG. 3) and PARP1 trapping (see FIGS. 8, 9) were analysed. U2OS cells treated with talazoparib show an enhanced retention of GFP-tagged PARP1 or PARP2 at induced DNA lesions, whereas cells treated with veliparib reveal overall less PARP2 recruitment, and only weak trapping at DNA lesions when compared to a DMSO-only control. In this example, the optimal imaging time to detect PARP1 and PARP2 trapping was determined to be around 15-30 minutes. This demonstrates that the described method can be used to measure differences in the effect of different compounds on protein trapping on the cell's genetic material, suitably DNA. Using an inhibitor against the chromatin remodeler ALC1 (ALCi), we demonstrate that proteins other than PARP enzymes, e.g. ALC1, can be trapped by an ALC1 inhibitor compound (see FIG. 4). Using this method, we examined the trapping of GFP-tagged ALC1 in the presence of ALCi-1 compared to a DMSO control. In this example, the imaging time for ALC1 could be reduced to around 5 minutes to see protein trapping effects. The disclosed method thus allows to screen for test compounds that directly act on proteins and investigate their ability to trap the inhibited protein. The disclosed method can be applied for screening of different compounds against many proteins, e.g. nuclear proteins, that can bind for example DNA or RNA.

Furthermore, the disclosed method can be used to measure indirect trapping effects of compounds, in which the compound induces trapping of a protein in an indirect manner. For example, we tested the indirect effect of an ALCi on trapping of PARP2 (see FIGS. 5 and 6). The method thus further allows to monitor the effect on protein trapping upon co-treatment with at least two compounds. This is shown by co-treatment using ALCi-2 and the PARPi veliparib: Co-treatment weakens the recruitment of GFP-PARP2 even further than veliparib treatment alone (FIG. 7). The method thus allows the detection of potential synergistic effects between two compounds, as increased protein trapping is an indicator for increased cellular cytotoxicity.

The disclosed methods can be used to analyse the effects of different compounds on their capability to trap a protein upon co-treatment with PARPi. This allows the detection of enhancing and/or differential effects. For example, this can allow the detection of enhancing the effect of a PARPi, such as veliparib, which is considered to be a poor PARPi trapping compound. The methods described herein are highly sensitive, and therefore allow the detection of compounds at low, non-toxic concentrations. This may aid their clinical use. Furthermore, the disclosed methods allow the detection of synergistic effects that can aid in overcoming resistance mechanisms frequently observed by PARPi. The disclosed methods may also be used to develop new combinations of compounds that induce a “HRDness” (HR deficiency) or “BRCAness” phenotype or use in the treatment of cancer. A loss of BRCA1 or BRCA2 indicates for BRCAness, including a defect in homologous recombination (HR) repair. Loss of these proteins leading to a HR defect defines vulnerability of multiple cancer types. Those cells are specifically sensitive to DNA damaging agents and PARPi, because of their error prone DNA repair. Recently, the term for BRCAness was expanded to include defects in replication fork protection, DNA damage checkpoint proteins and kinases regulating BRCA functions.

Additionally, the described method can be used to monitor the retention and release kinetics of proteins directly targeted by the at least one compound. Suitably, this can be used as a correlate for their efficacy. The disclosed methods can be used on both known drugs to measure their specificity as well as unknown compounds to determine their specificity and/or to improve methods to determine specificity of a compound. The disclosed methods can also be used in the development of many DDR-relevant therapeutic application based on genetic, epigenetic or other, functional loss of HR-repair pathways, including but not limited to BRCA1/2 deficiency.

The following Examples illustrate the disclosure described above; they are not, however, intended to limit the scope of the disclosure in any way. Other variants of the disclosure will be readily apparent to one of ordinary skill in the art and are encompassed by the appended claims.

### EXAMPLES

Eukaryotic cells were seeded onto 4/8-well Nunc Lab-Tek chambers (Thermo Fisher Scientific) in the corresponding cell culture medium, cultured over night at 37° C. and transfected with fluorophore-tagged protein plasmids using XtremeGENE HP DNA transfection reagent (Sigma). 24 h after transfection, the cells were treated with compounds of varying concentrations or a corresponding control (e. g. DMSO) for 1 hour.

The drug- and control-master-mixes were prepared in Leibovitz's L-15 media (+10% FBS). The cells were imaged using a Zeiss AxioObserver Z1 confocal spinning-disk microscope equipped with a sCMOS ORCA Flash 4.0 camera (Hamamatsu) through a C-Apo 63× water immersion objective lens. While imaging, the cells were kept at 37° C. in the absence of CO2. DNA damage was induced for 400 msec along a line of 88 pixels with a UV laser operated through a single-point scanning head (UGA-42 firefly, Rapp OptoElectronics). First, the fluorophore-tagged protein was imaged in the absence of compound (control). The retention time of different fluorophore-tagged proteins at DNA lesions varied between proteins. For PARP2, imaging time was about 15-30 minutes, whereas for ALC1, it was about 5 minutes. Therefore, the optimal imaging time for the protein of interest was determined by investigation of the trapping and release under control conditions (no treatment). The imaging time after irradiation should be long enough to see release of the protein from the damage site, indicated by a decrease in fluorescent signal. This gives a good range of detecting protein trapping. Subsequently, the fluorophore-tagged protein was measured in the presence of at least one compound. To allow measuring the impact of compound(s) on protein trapping, the laser-power can be optimized to prevent cell killing throughout the imaging experiment in the presence of the compound. Further, the overexpression of the tagged protein should be as low as possible, to mimic the endogenous level in the cell and avoid aberrant recruitment of the fluorophore-tagged protein. Cells with low, comparable nuclear fluorophore intensity allow measurement of protein trapping effects compared to the control, where no trapping is happening.

The accumulation of fluorophore-tagged proteins at micro-irradiation sites in the control and compound-treated conditions was quantified in Fiji/ImageJ using a custom-designed analysis pipeline (Rueden et al., 2017; Schindelin et al., 2012). First, single cells were isolated, and its positions were registered using StackReg (Thevenaz et al., 1998). The background fluorescence was measured by selecting an area of 70×70 pixels outside of the cell. The damage intensity was measured with a selection of 25×100 pixels that covered the induced DNA damage, and the fluorescence of the nucleus was obtained using AutoThresholding (“Li method”) of the entire nucleus. To quantify the recruitment of the fluorophore-tagged protein at the DNA lesion in each cell, a relative recruitment value was calculated for every time point: Damage(t)-Background(t)/Nucleus(t)-Background(t). This was set relative to the timepoint before damage (t0). To identify protein trapping, the relative recruitment of the fluorophore-tagged protein in the presence of a given compound was compared to the relative recruitment of the same protein in the absence of the compound (control). An enhanced fluorescent signal at the end of the imaged period tEND in comparison to the control condition was considered as enhanced protein trapping.

### Example 1

U2OS cells were treated with PARPi veliparib (10 μM) and talazoparib (100 nM) and transfected with GFP-PARP2. Grey signal indicates GFP-PARP2 in the nucleus. Kinetics of GFP-PARP2 recruitment to and dissociation from DNA lesion (induced by a 10% 355 nm laser) was measured over 15 minutes in the presence and absence of PARPi. Bright lines show recruitment of PARP2 to laser micro-irradiated damage sites. Images were taken 1 minute and 15 minutes after irradiation. Treatment with talazoparib shows enhanced retention of PARP2 at induced damage sites (“PARP trapping”), whereas treatment with veliparib leads to less recruitment of PARP2 to the damage site (see FIG. 3).

### Example 2

Wild-type U2OS cells were transiently transfected with GFP-ALC1. Kinetics of GFP-ALC1 recruitment to and dissociation from DNA lesion (induced by a 10% 355 nm laser) was measured over 5 minutes in the presence and absence of compound ALCi-1 (50 μM). Treatment with the compound shows enhanced retention of ALC1 at DNA damage sites compared to DMSO. 10 nuclei were analyzed in 1 biological replicate. The data are shown as mean+S.E.M. normalized to pre-damaged GFP intensity at micro-irradiation sites (see FIG. 4).

### Example 3

Wild-type U2OS cells were transiently transfected with GFP-PARP2. Kinetics of GFP-PARP2 recruitment to and dissociation from DNA lesion (induced by a 15% 355 nm laser) was measured over 30 minutes in the presence and absence of ALCi-2 (10 μM). Treatment with ALCi-2 shows enhanced retention of PARP2 at DNA damage sites compared to DMSO. 5-15 nuclei were analyzed in 1 biological replicate. The data are shown as mean+S.E.M. normalized to pre-damaged GFP intensity at micro-irradiation sites (see FIG. 5).

### Example 4

Wild-type U2OS cells were transiently transfected with GFP-PARP2. Kinetics of GFP-PARP2 recruitment to and dissociation from DNA lesion (induced by a 15% 355 nm laser) was measured over 30 minutes in the presence and absence of ALCi-2 (10 μM). Upper nucleus was treated with DMSO, lower nucleus was treated with ALCi-2 (10 μM) for 1 h. Timepoint 0 min. shows nuclei before micro-irradiation, timepoint 1 min. and 30 min. show nuclei after irradiation. Treatment with ALCi shows PARP2-trapping at DNA damage sites compared to DMSO (see FIG. 6).

### Example 5

Wild-type U2OS cells were transfected with GFP-PARP2. Kinetics of GFP-PARP2 recruitment to and dissociation from DNA lesion (induced by a 10% 355 nm laser) was measured over 15 minutes in the presence and absence of veliparib (10 μM) or a combination of veliparib (10 μM) and ALCi-2 (10 μM). 4-11 nuclei were analyzed in 1 biological replicate. The data are shown as mean+S.E.M. normalized to pre-damaged GFP intensity at micro-irradiation sites (see FIG. 7).

It will be understood that the invention has been described by way of example only and modifications may be made whilst remaining within the scope and spirit of the invention.

### Example 6

Wild-type U2OS cells were transiently transfected with PARP1-GFP. Kinetics of PARP1-GFP recruitment to and dissociation from DNA lesion (induced by a 10% 355 nm laser) was measured over 30 minutes in the presence and absence of PARPi (Olaparib (10 μM), Veliarpib (10 μM), Talazoparib 100 nM). Treatment with PARPi shows enhanced retention of PARP1 at DNA damage sites compared to DMSO, where treatment with talazoparib leads to most PARP1 retention. 5-20 nuclei were analyzed in 2 biological replicates. The data are shown as mean+S.E.M. normalized to pre-damaged GFP intensity at micro-irradiation sites (see FIG. 8).

### Example 7

Wild-type U2OS cells were transiently transfected with PARP1-GFP. Kinetics of PARP1-GFP recruitment to and dissociation from DNA lesion (induced by a 15% 355 nm laser) was measured over 30 minutes in the presence and absence of PARPi for 1 h (Olaparib (10 μM), Veliarpib (10 μM), Talazoparib 100 nM). Upper nucleus was treated with DMSO. Timepoint 0 min. shows nuclei before micro-irradiation, timepoint 1 min. and 30 min. show nuclei after irradiation. Treatment with PARPi shows PARP1-trapping at DNA damage sites compared to DMSO (see FIG. 9).

### REFERENCES

Gemeinsame Publikation des Zentrums far Krebsregisterdaten and der Gesellschaft der Epidemiologischen Krebsregister in Deutschland E. V. (2015-2016). Krebs in Deutschland far 2015/2016 (Nr. 12). Robert-Koch-Institut. https://www.krebsdaten.de/Krebs/DE/Content/Publikationen/Krebs_in_Deutschland/kid_2019/krebs_in_d eutschland_2019. pdf?_blob=publicationFile
Ahel, D., Horejsi, Z., Wiechens, N., Polo, S. E., Garcia-Wilson, E., Ahel, I., Flynn, H., Skehel, M., West, S. C., Jackson, S. P., et al. (2009). Poly(ADP-ribose)-dependent regulation of DNA repair by the chromatin remodeling enzyme ALC1. Science. 325, 1240-1243.
Gottschalk, A. J., Timinszky, G., Kong, S.E., Jin, J., Cai, Y., Swanson, S. K., Washburn, M. P., Florens, L., Ladurner, A. G., Conaway, J. W., et al. (2009). Poly(ADP-ribosyl)ation directs recruitment and activation of an ATP-dependent chromatin remodeler. Proc. Natl. Acad. Sci. 106, 13770-13774.
Kalousi, A., Hoffbeck, A.-S., Selemenakis, P. N., Pinder, J., Savage, K. I., Khanna, K. K., Brino, L., Dellaire, G., Gorgoulis, V. G. & Soutoglou, E. (2015). The Nuclear Oncogene SET Controls DNA Repair by KAP 1 and HP1 Retention to Chromatin. Cell Reports, 11(1), 149-163. https://doi.org/10.1016/j.celrep.2015.03.005
Kondrashova, O., Nguyen, M., Shield-Artin, K., Tinker, A. V., Teng, N. N. H., Harrell, M. I., Kuiper, M. J., Ho, G.-Y., Barker, H., Jasin, M., Prakash, R., Kass, E. M., Sullivan, M. R., Brunette, G. J., Bernstein, K. A., Coleman, R. L., Floquet, A., Friedlander, M., Kichenadasse, G., . . . Scott, C. L. (2017). Secondary Somatic Mutations Restoring RAD51C and RAD51D Associated with Acquired Resistance to the PARP Inhibitor Rucaparib in High-Grade Ovarian Carcinoma. Cancer Discovery, 7(9), 984-998. https://doi.org/10.1158/2159-8290.cd-17-0419
Lheureux, S., Lai, Z., Dougherty, B. A., Runswick, S., Hodgson, D. R., Timms, K. M., Lanchbury, J. S., Kaye, S., Gourley, C., Bowtell, D., Kohn, E. C., Scott, C., Matulonis, U., Panzarella, T., Karakasis, K., Burnier, J. V., Gilks, C. B., O'Connor, M. J., Robertson, J. D., . . . Oza, A. M. (2017). Long-Term Responders on Olaparib Maintenance in High-Grade Serous Ovarian Cancer: Clinical and Molecular Characterization. Clinical Cancer Research, 23(15), 4086-4094. https://doi.org/10.1158/1078-0432.ccr-16-2615
Lord, C. J., and Ashworth, A. (2012). The DNA damage response and cancer therapy. Nature 481, 287-294.
Luengo-Fernandez, R., Leal, J., Gray, A. & Sullivan, R. (2013). Economic burden of cancer across the European Union: a population-based cost analysis. The Lancet Oncology, 14(12), 1165-1174. https://doi.org/10.1016/s1470-2045(13)70442-x
Mok, M. C. Y., Campalans, A., Pillon, M. C., Guarne, A., Radicella, J. P., and Junop, M. S. (2019). Identification of an XRCC1 DNA binding activity essential for retention at sites of DNA damage. Scientific Reports, 9(1), 3095. https://doi.org/10.1038/s41598-019-39543-1
Murai, J., Huang, S.-N., Das, B. B., Renaud, A., Zhang, Y., Doroshow, J. H., Ji, J., Takeda, S. & Pommier, Y. (2012). Trapping of PARP1 and PARP2 by Clinical PARP Inhibitors. Cancer Research, 72(21), 5588-5599. https://doi.org/10.1158/0008-5472.can-12-2753
Murai, J., Huang, S.-Y. N., Renaud, A., Zhang, Y., Ji, J., Takeda, S., Morris, J., Teicher, B., Doroshow, J. H., and Pommier, Y. (2014). Stereospecific PARP trapping by BMN 673 and comparison with olaparib and rucaparib. Mol. Cancer Ther. 13, 433-443
Puumalainen, M. R., Lessel, D., Riithemann, P., Kaczmarek, N., Bachmann, K., Ramadan, K., and Naegeli, H. (2014). Chromatin retention of DNA damage sensors DDB2 and XPC through loss of p97 segregase causes genotoxicity. Nature Communications, 5(1), 3695. https://doi.org/10.1038/ncomms4695
Quigley, D., Alumkal, J. J., Wyatt, A. W., Kothari, V., Foye, A., Lloyd, P., Aggarwal, R., Kim, W., Lu, E., Schwartzman, J., Beja, K., Annala, M., Das, R., Diolaiti, M., Pritchard, C., Thomas, G., Tomlins, S., Knudsen, K., Lord, C. J., . . . Feng, F. Y. (2017). Analysis of Circulating Cell-Free DNA Identifies Multiclonal Heterogeneity of BRCA2 Reversion. Cancer Discov. 2017 Sep.;7(9):999-1005. doi: 10.1158/2159-8290.CD-17-0146.

1. A method for determining the capability of at least one compound for trapping of at least one protein of interest comprising the steps of
a. contacting a first cell comprising at least one detectably labelled protein with the at least one compound;
b. exposing said cell to conditions inducing damage to the cell's genetic material, suitably DNA and/or RNA;
c. detecting the at least one detectably labelled protein; and
d. identifying the at least one compound as capable of protein trapping when accumulation and/or residence time of the at least one protein of interest is increased;
wherein the compound is selected from an inhibitor of at least one of chromodomain-helicase-DNA-binding protein 1-like (ALC1); DNA repair and DNA damage response protein from Table 1; DNA replication proteins from Table 2; chromatin remodelers defined by presence of a SNF2-family ATPase domain; histone chaperone; transcription factors; RNA binding proteins; proteins in PARP pathways from Table 3.

2. The method according to claim 1, wherein the inhibitor targets chromodomain-helicase-DNA-binding protein 1-like (ALC1).

3. The method according to claim 1, comprising contacting a second cell comprising at least one detectably labelled protein with at least one control compound, wherein the at least one compound is considered capable of protein trapping when accumulation and/or residence time of the at least one protein is higher in the first cell than in the second cell.

4. The method according to claim 1, wherein the damage is induced by laser irradiation, suitably UV microirradiation.

5. The method according to claim 1, wherein the detecting is performed by live cell imaging.

6. A kit comprising:
a first cell comprising at least one detectably labelled protein and a second cell comprising at least one detectably labelled protein; and
instructions for using the first cell and the second cell in a method for screening of a test compound for capability to trap a protein, wherein the test compound is selected from an inhibitor of at least one of inhibitor of at least one of chromodomain-helicase-DNA-binding protein 1-like (ALC1); DNA repair and DNA damage response protein from Table 1; DNA replication proteins from Table 2; chromatin remodelers defined by presence of a SNF2-family ATPase domain; histone chaperone; transcription factors; RNA binding proteins; proteins in PARP pathways from Table 3.

7. A screening assay comprising:
a. Exposing a plurality of cell samples to DNA damage conditions, wherein said samples comprise at least one protein; and
b. Determining protein trapping of the protein in a first cell sample in the presence of at least one test compound relative to a second cell sample not comprising said at least one test compound,
c. wherein a test compound contained in the first cell sample in which the detectably labelled protein exceeds the accumulation and/or residence time of the protein in the second cell sample is identified as a candidate compound for protein trapping,
wherein said test compound is selected from an inhibitor of at least one of chromodomain-helicase-DNA-binding protein 1-like (ALC1); DNA repair and DNA damage response protein from Table 1; DNA replication proteins from Table 2; chromatin remodelers defined by presence of a SNF2-family ATPase domain; histone chaperone; transcription factors; RNA binding proteins; proteins in PARP pathways from Table 3.

8. The assay of claim 7, wherein DNA damage conditions are induced by laser irradiation.

9. The assay of claim 7, wherein the at least one protein is detectably labelled.

10. The assay of claim 9, wherein the detectably labelled protein is detected by live cell imaging.

11. The method of claim 1, the kit of claim 6, or the assay of claim 7, wherein the protein of interest is selected from chromodomain-helicase-DNA-binding protein 1-like (ALC1); DNA repair and DNA damage response protein from Table 1; DNA replication proteins from Table 2; chromatin remodelers defined by presence of a SNF2-family ATPase domain; histone chaperone; transcription factors; RNA binding proteins; proteins in PARP pathways from Table 3.

12. The method of claim 1, wherein the at least one detectably labelled protein and the at least one inhibited protein are the same or are different.

13. The method of claim 1, wherein the detectably labelled protein is selected from ALC1 and PARP, and/or the detectable label is a fluorescent label or a quantum dot label.

14. (canceled)

15. The method of claim 1, the kit of claim 6, or the assay of claim 7, wherein said cells are human cells.

16. The kit of claim 6, wherein the protein of interest is selected from chromodomain-helicase-DNA-binding protein 1-like (ALC1); DNA repair and DNA damage response protein from Table 1; DNA replication proteins from Table 2; chromatin remodelers defined by presence of a SNF2-family ATPase domain; histone chaperone; transcription factors; RNA binding proteins; proteins in PARP pathways from Table 3.

17. The assay of claim 7, wherein the protein of interest is selected from chromodomain-helicase-DNA-binding protein 1-like (ALC1); DNA repair and DNA damage response protein from Table 1; DNA replication proteins from Table 2; chromatin remodelers defined by presence of a SNF2-family ATPase domain; histone chaperone; transcription factors; RNA binding proteins; proteins in PARP pathways from Table 3.

18. The kit of claim 6, wherein the at least one detectably labelled protein and the at least one inhibited protein are the same or are different.

19. The assay of claim 7, wherein the at least one detectably labelled protein and the at least one inhibited protein are the same or are different.

20. The kit of claim 6, wherein the detectably labelled protein is selected from ALC1 and PARP, and/or the detectable label is a fluorescent label or a quantum dot label.

21. The assay of claim 7, wherein the detectably labelled protein is selected from ALC1 and PARP, and/or the detectable label is a fluorescent label or a quantum dot label.
